(19) United States
(12) Patent Application Publication
(10) Pub. No.: US 2013/0121461 A1
(43) Pub. Date: May 16, 2013

(54) METHOD OF MANAGING A WEIGHT CONDITION IN AN ANIMAL

(71) Applicants: Philip W. Toll, Valley Falls, KS (US); Inke Paetau-Robinson, Auburn, KS (US); Claudia A. Kirk, Louisville, TN (US)

(72) Inventors: Philip W. Toll, Valley Falls, KS (US); Inke Paetau-Robinson, Auburn, KS (US); Claudia A. Kirk, Louisville, TN (US)

(73) Assignee: HILL'S PET NUTRITION, INC., Topeka, KS (US)

(21) Appl. No.: 13/520,191

(22) PCT Filed: Jan. 6, 2011
(86) PCT No.: PCT/US11/20359

§ 371 Date: Jul. 2, 2012

Related U.S. Application Data
(60) Provisional application No. 61/292,652, filed on Jan. 6, 2010.

(51) Int. Cl. A61B 5/00; A61B 6/00; A61B 5/107

(52) U.S. Cl. CPC A61B 5/4872; A61B 5/7278; A61B 5/1072; A61B 6/508
USPC 378/53; 600/587

(57) ABSTRACT

A methodology of managing a weight condition of a companion animal by determining body fat composition of the companion animal and an appropriate weight loss regimen based on the body fat percentage is provided. More specifically, described herein is a clinically useful tool and methodology to apply to over-weight and obese animals for use in managing a weight condition of the overweight or obese animal by determining the body fat percentage of the animal and providing a weight loss regimen.

### CROSS REFERENCE TO RELATED APPLICATIONS

This application claims priority to U.S. Provisional Patent Application No. 61/292,652, filed on Jan. 6, 2010, which is incorporated herein by reference.

### BACKGROUND

The embodiments described herein relate to a methodology of assessing body fat and determining an appropriate weight loss regimen for companion animals. More specifically, described herein is a clinically useful tool and methodology to apply to overweight and obese animals.

Obesity is on the rise in the United States, and not only in humans. In 2008, a companion animal obesity study by the Association for Companion Animal Obesity Prevention concluded that an estimated 84 million U.S. dogs and cats are overweight or obese, accounting for approximately 50% of dogs and cats. Moreover, an estimated 10% of dogs and an estimated 18% of cats are obese. In fact, obesity is considered one of the most common forms of malnutrition occurring in dogs.

Generally, companion animals such as canines and felines weighing more than 15% of their ideal body weight are considered overweight or obese. Overweight animals generally have an excess of body adipose tissue. The most common cause of an animal being overweight is an over consumption of food that results in an excess intake of calories. Studies have shown that fat animals are significantly more at risk for diseases such as arthritis, heart disease, respiratory disease, diabetes, bladder cancer, hypothyroidism, and pancreatitis.

As companion animals become more and more obese, the difficulties presented to the veterinarian or animal practitioner become increasingly apparent. One difficulty realized by many veterinarians is the need to accurately prescribe the amount of food that the companion animal owner should feed to the companion animal in order to attain the optimum level of health for the companion animal. In order to accurately prescribe the amount of food that the companion animal owner should feed the companion animal, the veterinarian must first accurately assess the energy needs of the animal. Likewise, in order to accurately prescribe the energy needs of the animal, the veterinarian must accurately determine the percentage body fat of the animal.

Thus, the process of prescribing the proper amount of food for an appropriate weight loss regimen is ultimately dependent upon, among other things, the accurate calculation of body fat percentage. The more error in the calculation of body fat percentage, the more incorrect the caloric assessment will be.

Currently, the technique of body condition scoring (BCS) is the most accessible and popular method for estimating obesity in companion animals. This method is accessible and popular because of its simplistic use of physical criteria that are easily measurable by the veterinarian or animal practitioner. Under the BCS method, physical examination, visual observation, and palpation may be used to assign a body condition score. The body condition score is a semi-quantitative assessment of body fat with a range of categories from lean to severely obese. The estimates of the BCS method, although inexact, have been confirmed to roughly correlate to the actual body fat percentage as determined by dual-energy X-ray absorptiometry (DEXA).

However, the BCS method is largely ineffective in many instances. Because the BCS method applies the same testing criteria, it attempts a one-size-fits-all solution to a challenging dynamic problem. Additionally, the specific physical parameters that should be measured in order to clinically assess a companion animal's body fat percentage may not be equivalent in each situation. Although anthropomorphic measurements such as skinfold measurements have historically been applied to estimate body fat percentage in humans, these types of measurements have been found to be less effective in companion animals. In essence, the diagnostic procedures for assessing body fat that are currently available to practicing veterinarians and animal practitioners do not remedy the problems associated with the current flawed techniques. For example, while the body fat in humans can be closely estimated using skinfold calipers, the canine triceps is not as cooperative.

While rudimentary methods such as the BCS method are more accurate for companion animals with a low amount of fat, these multiple body condition scoring methods are insufficient to estimate the body fat over the range of obese companion animals. Because an accurate assessment of body fat in an animal is a prerequisite to establishing ideal weight and calculating an accurate caloric dose for weight loss, the margin of error is compounded in the typical procedures for prescribing a weight loss regimen.

Morphometric measurements have been used in dogs and cats, but little has been published comparing objective body measurements with body fat. In part, this is due to the fact that companion animals deposit and store fat subcutaneously in various locations, including the thoracic, lumbar, and coccygeal areas as well as intra-abdominally. When companion animals are subject to weight gain, the pelvic circumference usually changes the most. While specific measurements of the pelvic circumference have at times been used to estimate body fat percentage, this method is also lacking in accuracy and precision.

Because the current methods for estimating body fat percentage of companion animals are often ineffective, the present invention attempts to advance the tools available to the veterinarian and animal practitioner based on objective criteria and statistical analysis. Accordingly, a methodology of assessing body fat and for determining an appropriate weight loss regimen for companion animals is provided. In accordance with the present invention, a method is additionally provided to assist practitioners with practical diagnostic tools to determine body fat and ideal body weight in companion animals, particularly in overweight and obese companion animals. Using this information, the present invention also provides a simple means of calculating the energy needs of an animal and an effective food dose for weight loss therapy.

### BRIEF SUMMARY OF THE INVENTION

In one aspect of the present invention, a method of managing a weight condition in a companion animal using tools to estimate the body fat percentage of the companion animal is provided. The method includes using the body fat percentage to provide an effective weight loss regimen for the companion animal. Further, the method involves determining the ideal body weight of the companion animal, the daily feeding amount to reduce the companion animal's weight to a desirable level, and the expected weight loss of the companion animal, provided the daily feeding regimen is followed.

In a further aspect of the invention, the formula for body fat assessment is determined by regression analysis. Using DEXA results or similar reliable methods to determine the actual percentage body fat or lean body mass, physical data may be measured and descriptive data may be used to correlate the data and develop equations to predict percentage body fat or lean body mass based on the measured and descriptive data.

In still a further aspect of the invention, the formula for body fat assessment is divided into two separate formulas: one formula for animals with body weight less than or equal to a threshold amount, and a separate formula for animals with body weight greater than a threshold amount.

In still a further aspect of the present invention, the animals are dogs and the threshold amount is 40 pounds.

In still a further aspect of the invention, the animals are cats.

In still a further aspect of the invention, a method is provided whereby a practitioner may utilize a spreadsheet, program, or similar tool to enter descriptive and measurement information in order to automatically calculate the percentage body fat, the ideal body weight, the resting energy requirements, the food dose amounts, and any other information relating to the weight loss program for the companion animal.

It is to be understood that both the foregoing general description of the invention and the following detailed description are exemplary, but are not restrictive, of the invention.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates a high level flow chart of a method of assessing body fat and determining an appropriate weight loss regimen;

FIG. 2 illustrates exemplary methods for the estimation of body fat percentage in companion animals;

FIG. 3 illustrates a high level flow chart of a method of first using a reliable process to determine the body fat of a group of animals, and then measuring physical data in order to apply regression analysis to formulate best-fit equations for the clinically-friendly calculation of body fat percentage and the ultimate prescription of a weight loss regimen;

FIG. 4 illustrates exemplary input parameters and output parameters for a target weight and food dose calculator for dogs less than or equal to 40 lbs.;

FIG. 5 illustrates exemplary input parameters and output parameters for a target weight and food dose calculator for dogs greater than 40 lbs.

### DETAILED DESCRIPTION

A methodology of managing a weight condition in a companion animal is herein provided. The methodology is particularly useful for more accurate assessment in animals having greater than average body fat.

An exemplary embodiment of the present invention is a method of managing a weight condition in a companion animal comprising determining the estimated body fat percentage of the companion animal and providing an effective weight loss regimen for the companion animal based on the estimated body fat percentage. The method further comprises determining an ideal body weight for the companion animal, determining a daily feeding regimen to prescribe to the companion animal in order to reduce the companion animal's weight to the ideal body weight, and determining a rate of expected weight loss of the companion animal, provided the daily feeding regimen is followed.

In some embodiments, the method comprises a food composition wherein the food composition comprises protein, fat, fiber and carbohydrate.

In one embodiment, the method comprises determining the estimated body fat percentage of the companion animal by the Body Fat Scoring (BFS) method, wherein a visual and palpate assessment of an animal's body fat is made and the results of the visual and palpate assessment are used to assign a body fat index score to the animal. The visual and palpate assessment may include the following: amount of face cover on the head and neck, prominence of bony structures in the face, distinction between the head and shoulder, scruff tightness and fat amount on neck, amount of pectoral fat, prominence and ease of palpation for the sternum, scapula and ribs, inguinal fat pad on the abdomen, ease of palpation of abdominal contents and overall body assessment including the shape from the side, shape from above, and stance. The body fat index score is generally understood to be a whole number that is an estimate of the body fat percentage for the animal. This method may also comprise a subjective assessment of the physical criteria observed during the visual and palpate assessment, with each assessment being assigned a particular number of points. The points may then be totaled to arrive at the estimated body fat index score.

In one embodiment, the method comprises determining the estimated body fat percentage of the companion animal by the Body Fat Prediction (BFP) method, wherein biological information and physical measurements are used to arrive at an estimated body fat percentage. Such biological information and physical measurements may include body weight, age, gender and neuter status with measurements such as height, length, leg length, foot pad size, etc.

In some embodiments, the method also includes determining the estimated body fat percentage of the companion animal by a spreadsheet, computer program, database, or similar tool developed to receive input information and to automatically calculate the estimated percentage of body fat, the ideal body weight, the resting energy requirements (RER), and the food dose amounts.

An exemplary embodiment of the present invention is a method of managing a weight condition of a companion animal comprising using methods to determine the actual percentage of body fat or lean body mass of a companion animal; using measured physical data of the companion animal and descriptive data of the companion animal to apply regression analysis based on the actual percentage of body fat or lean body mass; and deriving one or more equations based on the regression analysis, the one or more equations for predicting the percentage body fat or lean body mass of the companion animal based on measured physical data and descriptive data of the new companion animal. In a preferred embodiment, the method to determine the actual percentage of body fat or lean body mass of a companion animal is dual-energy X-ray absorptiometry (DEXA).

The method also includes where the one or more equations are two separate equations, the first equation to be applied to companion animals with body weight less than a threshold amount, and the second equation to be applied to companion animals with body weight greater than a threshold amount.

The method also includes where the companion animals are dogs, and the threshold amount is 40 pounds.

In still a further aspect of the invention, the animals are cats.

In a further embodiment, the present invention provides a kit comprising in separate containers in a single package a (1) means for communicating information about or instructions for a method of assessing a companion animal comprising determining the estimated body fat percentage of the companion animal and providing an effective weight loss regimen for the companion animal and (2) a food fat used to promote weight loss in the companion animal.

Although exemplary tools are described herein to obtain an estimate of the body fat percentage of the animal, particularly for use with obese animals, it is readily understood by those having skill in the art that various methods may be utilized for estimating the body fat percentage.

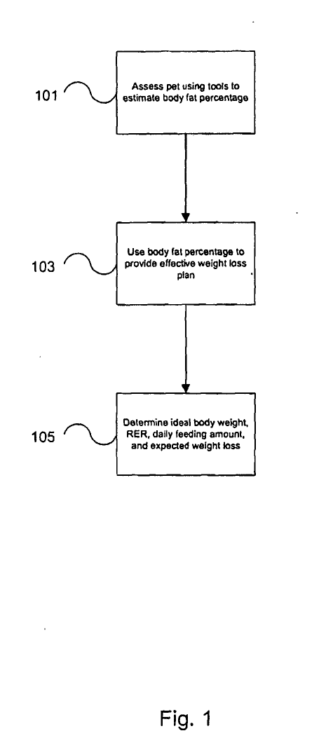

According to FIG. 1, the first step in the process is to assess the companion animal using tools to estimate the body fat percentage 101. As further described below, this can be performed in a variety of fashions. An exemplary methodology described below utilizes body fat assessment tools and a weight loss calculator or similar tool. The companion animal is assessed using criteria to provide a body fat index or score. The body fat index or score may either be based on an estimate of the percentage body fat of the animal, or the actual percentage body fat of the animal. This number is then entered into a calculator or similar tool, which in turn provides the information necessary for an effective weight loss regimen 103. This information may include the ideal body weight of the animal, the resting energy requirement (RER), the daily feeding amount, and the expected weight loss 105.

In animal weight assessment, once the body fat percentage has been estimated 101, the estimated body fat percentage may be used to estimate the RER and the ideal body weight of the animal 105. Using the current BCS method that is applied to normal-weight animals, the process has the undesirable result of over-estimating the daily caloric need in animals that have excess body fat. As the body fat of the animal increases, the over-estimation of daily caloric need becomes greater and greater. Therefore, the current process further complicates the problem that it was initially designed to address.

The over-estimation resulting from application of the BCS method was recently discovered in an initial study leading to the development of the present invention. More fully described below, the initial study demonstrated that current methods of estimating ideal body weight for weight-loss feeding are largely inaccurate for dogs having greater than 45% body fat.

A further downside to the BCS method is the more obese the animal, the less accurate the method. In fact, the BCS method becomes increasingly inaccurate for animals with body fat percentages above 45%. In part, this is because BCS was designed primarily to assess dogs with body fat percentages at less than 45%. Because of the increasing number of dogs with high-percentages of body fat, BCS as a one-size-fits-all method is becoming less and less effective. For instance, many obese dogs currently have body fat percentage at a level above 50%, for which the BCS method is largely ineffective.

An element of the initial study demonstrated that current methods (i.e., BCS) of estimating ideal body weight for weight loss feeding are inaccurate in dogs having more than 45% body fat. The two major limitations of the current methods of assessing body fat is that (1) precision and accuracy are highly dependent on the training and skill of the individual doing the assessment, and (2) the current body condition scoring scales do not differentiate between different levels of obesity. For example, in the BCS 5 point scale, all dogs with greater than 35% body fat fall into a single score of 5. This has the undesirable effect that a dog with 60% body fat and a dog with 36% body fat both receive the same score. The fatter the dog, the more overestimation of ideal body weight and feeding amount, and therefore the slower and more ineffective the weight loss program.

The initial study compared the accuracy of using body fat percentages to the 5 and 9 point BCS systems for estimating ideal body weight and RER in the dogs. Once a BCS value was assigned by an animal practitioner, the median body fat percentage for each score was used to estimate ideal body weight and RER. Based on the results of the DEXA scans, the body fat ranged from 28.3% to 63.7%, with a mean of 45.9%. In order to assess the accuracy of BCS for moderately versus morbidly obese dogs, the dogs were divided into two groups. The first group had less than 45% body fat, and the second group had greater than 45% body fat. There were 15 dogs in the first group and 21 dogs in the second group.

Compared to DEXA, estimations of ideal body weight were significantly higher using the 5 (23.0 vs. 19.2 kg) and 9 (21.1 vs. 19.2 kg) point BCS in dogs with body fat greater than 45% (p<0.001) but did not differ in dogs with less than 45% (p>0.05).

The results of the above study therefore demonstrate that current BCS systems provide good estimates of ideal body weight and RER in dogs with less than 45% body fat, but are inadequate for calculating RER and ideal body weight in morbidly obese dogs with body fat greater than 45%.

Adding to the problem of assessing the body fat percentage is the error associated with estimating the RER. For an animal weight loss program to remain effective, the daily caloric intake of the animal should be restricted below the level required to maintain the current body weight. In normal-weight animals, the calculation of daily caloric need may be based on the body weight of the animal. However, applying the same approach to above-weight animals can have negative consequences, including over-estimation of the daily caloric needs of the animal.

In addition, a further study described below suggests that DEXA (or equivalent techniques) may be used in combination with known morphometric measurements and basic biological information to use statistical analysis to formulate a best-fit equation, the best-fit equation being appropriate for determining an effective weight loss regimen for any domestic companion animal.

The following is a summarizing description of how morphometric measurements may be taken. A person having ordinary skill in the art will realize that any similar manner of measuring physical attributes may be properly understood as equivalent, and the following is merely exemplary and non-limiting in nature. For instance, body length may be measured by using a Seca measuring rod to measure from the sternum to seat bone/rectum with the companion animal in a normal standing position and head pointing straight forward. Front height may be measured using the Seca floor height rod for measuring the standing height at the shoulder. Rear height may be measured using a Seca floor height rod for measuring the standing height at the hip. Thoracic circumference may be measured using a tailor's tape to wrap the tape tightly around the rib cage at the heart girth when measuring. The pelvic circumference may be measured using a tailor's tape to wrap tightly around the loin area just in front of the knee.

Next, provided herein is a description of the leg measurements. The hind leg length may be measured using a metal tape measure to measure the length of the hind leg from the central foot pad to the dorsal tip of the calcaneal process. Hind leg calcaneus width may be measured using a digital caliper to measure the width of the calcaneus. The hind leg central foot pad width may be measured using a digital caliper and laying the micrometer flat into the foot at the base of the pad. Hind leg central foot pad length may be measured using a digital caliper and laying the micrometer flat into the foot at the base of the pad. The front leg measurements are similar to the hind leg measurements, except the front legs are measured instead of the hind legs.

Head measurements may be provided as follows. The cranial length may be measured using a tailor's tape to measure from the exterior occipital protuberance to the medial canthus of the eye. The facial length may be measured using a tailor's tape to measure from the medial canthus of the eye to the tip of the nose. Head circumference may be measured using a tailor's tape to measure the circumference between the eyes and the ears at the widest part of the head. Finally, head width may be measured using the Seca measuring rod to measure between the eyes and ears.

After the measurements are recorded, multiple regression analysis may be applied using the DEXA results in order to develop regression equations for the prediction of lean body mass and fat mass from the measured body data and input descriptive data. The descriptive data can include anything from body weight, species, age, gender, neuter status, etc.

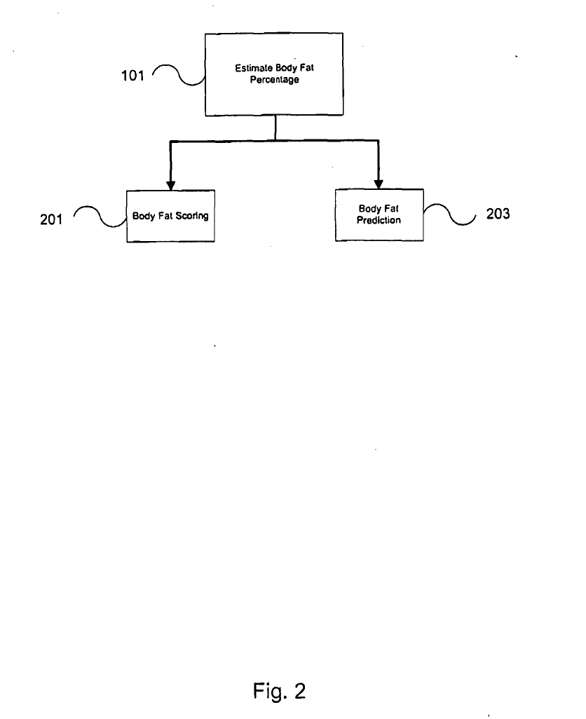

As described herein, two basic types of tools may be used to obtain an estimate of the body fat percentage of the animal. In accordance with FIG. 2, an exemplary method is provided called the Body Fat Scoring (BFS) method 201. In the BFS method 201, a visual and palpate assessment of body fat is made. This method uses the observations of a trained individual to assign a body fat index score to an individual animal. The body fat index score is generally understood to be a whole number that is an estimate of the percentage of body fat for that animal.

In one execution of the BFS method 201, the animal is assessed using a chart that lists the characteristics for each body fat index category and is assigned a corresponding score. For instance, the body fat index score of 10 may indicate a range of 5-15% of body fat. The score of 10 requires that the ribs are prominent, easily felt, and contain little fat cover. The score of 10 also requires that the shape of the dog from above is a marked hourglass shape; the shape from the side is a pronounced abdominal tuck; the shape from behind is prominent bones and an angular contour; the tail base contains prominent bony structures, is easily felt, and contains little fat cover. The following table illustrates extensive categories of the body fat index score.

TABLE 1
Body Fat Index

| BFI Index | BFI Index | BFI Index | BFI Index | BFI Index | BFI Index |
|---|---|---|---|---|---|
| 10 | 20 | 30 | 40 | 50 | 60 |
| 5-15% BF | 15-25% BF | 25-35% BF | 35-45% BF | 45-55% BF | 55-65% BF |
| Ribs | Ribs | Ribs | Ribs | Ribs | Ribs |
| Prominent; | Slightly | Slightly to not | Not prominent; | Not prominent; | Not prominent; |
| Easily felt; | prominent; | prominent; can | very difficult to | extremely | impossible to |
| Little fat cover | easily felt; thin | be felt; | feel; thick fat | difficult to feel; | feel; extremely |
| | fat cover | moderate fat | cover | very thick fat | thick fat cover |
| | | cover | | cover | |
| Above Shape | Above Shape | Above Shape | Above Shape | Above Shape | Above Shape |
| Marked | Well | Detectable | Loss of lumbar | Markedly | Extremely |
| hourglass shape | proportioned | lumbar waist | waist; | broadened back | broadened back |
| | lumbar waist | | broadened back | | |
| Side Shape | Side Shape | Side Shape | Side Shape | Side Shape | Side Shape |
| Pronounced | Abdominal tuck | Slight | Flat to bulging | Marked | Severe |
| abdominal tuck | present | abdominal tuck | abdomen | abdominal | abdominal |
| | | | | bulge | bulge |
| Behind Shape | Behind Shape | Behind Shape | Behind Shape | Behind Shape | Behind Shape |
| Prominent | Clear muscle | Losing muscle | Rounded to | Square | Square |
| bones; angular | definition; | definition; | square | appearance | appearance |
| contour | smooth contour | rounded | appearance | | |
| | | appearance | | | |
| Tail Base | Tail Base | Tail Base | Tail Base | Tail Base | Tail Base |
| Prominent bony | Slightly | Slightly to not | Bony structures | Bony structures | Bony structures |
| structures; | prominent bony | prominent bony | are not | are not | are not |
| easily felt; little | structures; | structures; can | prominent; very | prominent; | prominent; |
| fat cover | easily felt; thin | be felt; | difficult to feel; | extremely | impossible to |
| | fat cover | moderate fat | thick fat cover | difficult to feel; | feel; extremely |
| | | cover | | very thick fat | thick fat cover; |
| | | | | cover; fat | large fat dimple |
| | | | | dimple or fold | or fat fold |
| | | | | present | |

As expressed by the above table, each body fat index category covers a 10 point range in percentage of body fat. The body fat index score may then be entered into a weight loss calculator to obtain the ideal weight and feeding information.

As will be readily understood by a person having ordinary skill in the art, a way to describe this method is the subjective assessment of physical criteria based on multiple physical locations on the animal, with each assessment assigning a particular number of points. Once all the locations of the animal have been assessed, the points may be totaled to arrive at the estimated body fat index score. Then, the body fat index score may be entered into the weight loss calculator to obtain the ideal weight and feeding information.

The following table describes an exemplary body fat index scoring point system. When each of the criteria is evaluated by visual inspection and palpation, the total points may be combined.

TABLE 2
Body Fat Index Scoring Point System

| | Description | | | | | | |
|---|---|---|---|---|---|---|---|
| | Points | | | | | | |
| | | | | | | | Point |
| Criteria | 4 | 6 | 8 | 10 | 12 | 14 | Assignment |

| | | | | | | | |
|---|---|---|---|---|---|---|---|
| 1 | Ribs& | Thin | Minimal to | Moderate | Thick to | Very thick | Extremely |
| | Tail Base - | | moderate | to thick | very thick | to | thick |
| | Fat | | | | | extremely | |
| | Cover | | | | | thick | |
| 2 | Ribs & | Easily felt | Can be felt | Difficult to | Very | Extremely | Impossible |
| | Tail Base - | | | feel | difficult to | difficult to | to feel |
| | Palpation | | | | feel | feel | |
| 3 | Shape | Well | Detectable | Loss of | No lumbar | Lumbar | Severe |
| | from | proportioned | lumbar | lumbar | waist; | bulge; | lumbar |
| | above | lumbar | waist | waist; | markedly | markedly | bulge; |
| | | waist | | broadened | broadened | broadened | markedly |
| | | | | back | back | back | broadened |
| | | | | | | | back |
| 4 | Shape | Abdominal | Abdominal | Slight to | Slight to | Severe | Very |
| | from the | tuck present | tuck present | no | moderate | abdominal | severe |
| | side | | | abdominal | abdominal | bulge | abdominal |
| | | | | tuck | bulge | | bulge |
| 5 | Shape | Clear | Losing | Rounded | Square | Square | Square |
| | from | muscle | muscle | to square | appearance; | appearance; | appearance; |
| | behind | definition; | definition; | appearance | small to | large fat | large fat |
| | | smooth | rounded | | moderate | dimple | fold at tail |
| | | contour | appearance | | fat dimple | | base |

Total Points (BFI)

Improving the current BCS scale with the above BFS scale may provide for the correct food dose prescription for weight loss in severely obese companion animals. Moreover, a numerical point assignment methodology that allows the animal practitioner to enter data may be easily programmed into a Microsoft Excel spreadsheet, Microsoft Access database, or a similarly devised tool.

A second exemplary method for assessing the body fat percentage of the animal is the body fat prediction (BFP) method 203. The BFP method 203 is the above described method that uses basic biological information and simple physical measurements to predict body fat and ideal body weight. This method can be described as formulating equations by using regression analysis techniques explained above, in order to predict the percentage of body fat or lean body mass based on physical data attainable by the practicing veterinarian. For instance, descriptive information such as body weight, age, gender, and neuter status may be combined with simple measurements (such as height, length, leg length, foot pad size, etc.) in order to arrive at an estimated body fat percentage.

According to an embodiment of the present invention, regression equations may be used to predict either lean body mass or fat mass. The percentage of body fat can then be calculated using either the lean body mass or the fat mass and the total body weight. The basic data required for body fat prediction may be entered into a BFP calculator which provides a tool for calculating the percentage of body fat and other body fat variables. The percentage of body fat can be entered into the same weight loss calculator as above or the weight loss calculations may be automatically incorporated into the BFP calculator. The BFP method 203 therefore provides an accurate and objective measurement, while maintaining a suitable format for the clinical setting.

The ideal body weight and food dose calculator may also be provided as a tool for calculating the RER and amount of food to daily feed the animal. For instance, the ideal weight calculator may receive as input the BFS score and the current body weight of the animal. Alternatively, the ideal weight calculator may receive as input the descriptive information and equation parameters for the BFP method 203. As an output, the ideal body weight calculator may determine the ideal weight of the animal, the RER calculation (i.e. kcal/day), and the amount of food to feed the animal. In addition, the ideal body weight and food dose calculator may determine the percentage of lean body mass and the amount of lean body mass, and alternatively display this information in spreadsheet format to the animal practitioner.

An alternative embodiment of the present invention may separate the spreadsheets for determining percent body fat and ideal body weight and determining the food dose based on the calculated information and the type of food. Likewise, separate spreadsheets may be used for any category of animal to which separate equations are to be applied. For instance, a table may be used to input morphometric measurements for dogs less than or equal to 40 pounds, and a separate table may be used to input morphometric measurements for dogs greater than 40 pounds. In this manner, separate equations may run the backend process whereby the output variables are calculated.

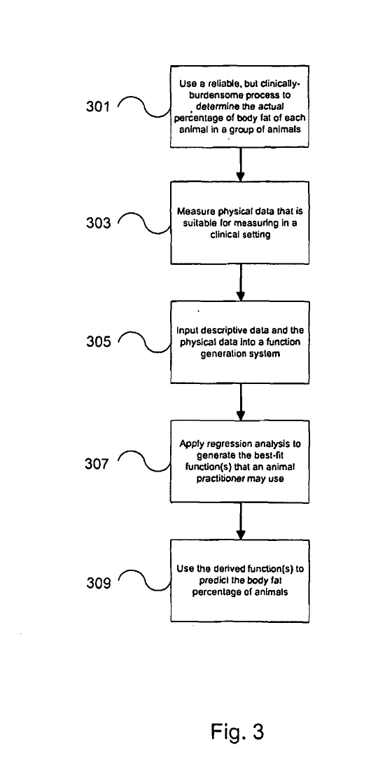

In FIG. 3, it is shown that for an exemplary process to be applied, one must first use a reliable, but clinically-burdensome process to determine the actual percentage of body fat of each animal in a group of animals 301. Next, the user may measure physical data that is suitable for measuring in a clinical setting 303. This allows the user to input the physically measured data, as well as descriptive data 305, in order to derive a function suitable for the clinical setting. Regression analysis may then be used to generate the best-fit function(s) that the animal practitioner may use for the clinical setting 307. Finally, the derived function(s) may be used to predict the body fat percentage of animals 309.

Using a tool to predict the body fat percentage of an animal, the animal practitioner may then estimate ideal body weight, calculate the RER, and determine a daily food regimen for the animal in order to meet the ideal body weight goals.

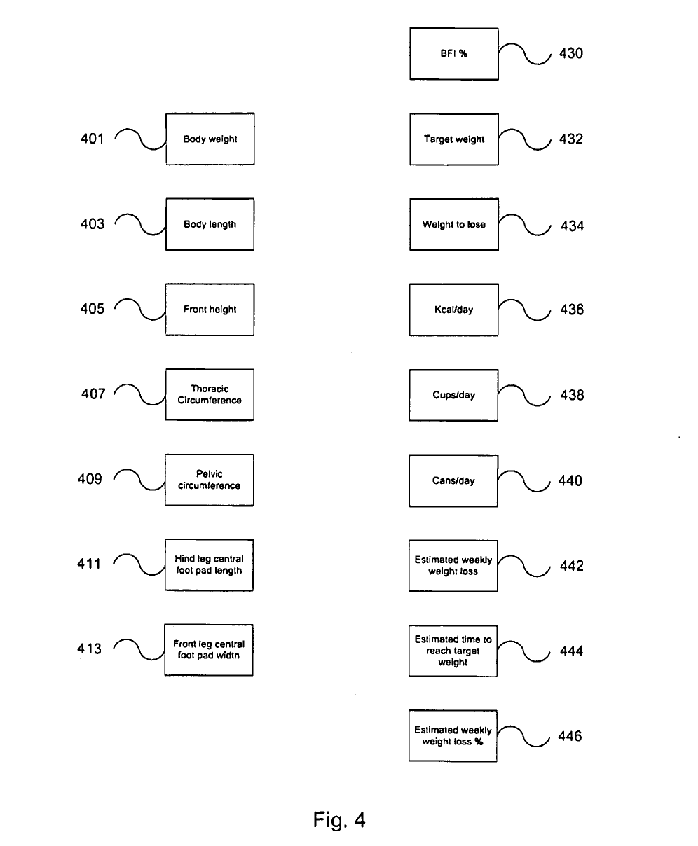

FIG. 4 shows exemplary input and output parameters that may utilized in a preferred embodiment of a spreadsheet for dogs less than or equal to 40 lbs. Body weight 401, body length 403, front height 405, thoracic circumference 407, pelvic circumference 409, hind leg central foot pad length 411, and front central foot pad width 413 are the parameters input into the spreadsheet in accordance with the above-described best fit algorithm for dogs less than or equal to 40 lbs. Accordingly, the output parameters include BFI % 430, target weight 432, weight to lose 434, Kcal/day 436, Cups/day 438, Cans/day 440, estimated weekly weight loss 442, estimated time to reach target weight 444, and the estimated weekly weight loss % 446.

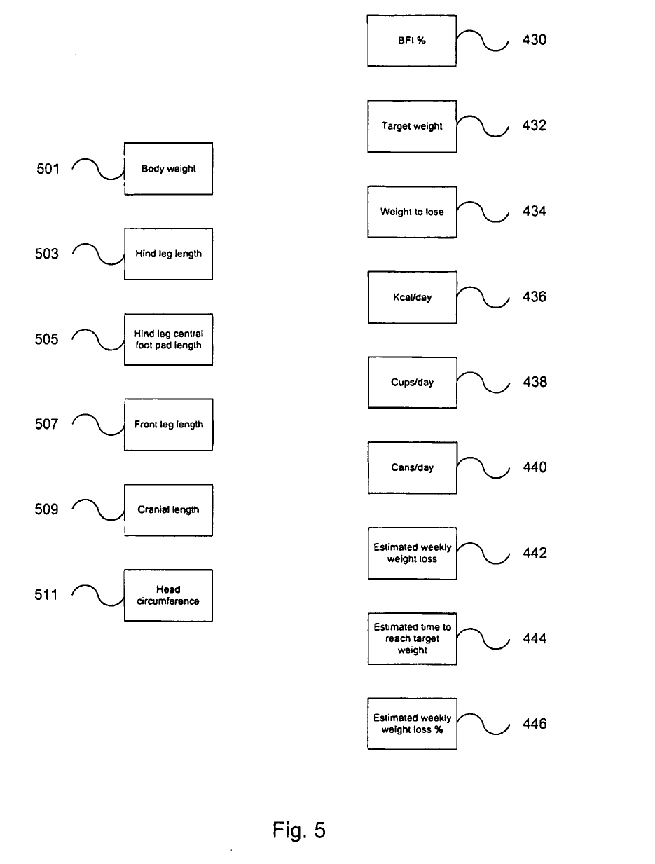

FIG. 5 shows exemplary input and output parameters that may utilized in a preferred embodiment of a spreadsheet for dogs greater than 40 lbs. Body weight 501, hind leg length 503, hind leg central foot pad length 505, front leg length 507, cranial length 509, and head circumference 511 are the parameters input into the spreadsheet in accordance with the above-described best fit algorithm for dogs greater than 40 lbs. Similarly, the output parameters include BFI % 430, target weight 432, weight to lose 434, Kcal/day 436, Cups/day 438, Cans/day 440, estimated weekly weight loss 442, estimated time to reach target weight 444, and the estimated weekly weight loss % 446.

Whether the BFS method or the BFP method is utilized to estimate the percentage of body fat of the animal, one should immediately realize improved dietary food prescriptions based on caloric intake, especially in overweight and obese animals.

### EXAMPLES

### Example 1

Thirty-six adult dogs with body composition ranging from overweight to morbidly obese were evaluated. The following measurements and procedures were conducted: body weight, palpation and visual assessment, digital photographs (front, rear, side and from above), body size and shape measurements, radiographs (head, thoracic and pelvic), and DEXA.

Lean body mass, fat mass and percent body fat were determined by DEXA. This data was used to evaluate other methods by providing the dependent variables to predict body composition (lean body mass, fat mass and percent body fat) by using independent variables obtained from morphometric measurements, skeletal measurements, body weight, age, gender, and neuter status. In this manner, equations to predict lean body mass, fat mass, and percent of fat were derived. Two separate models were applied. The first model was derived from the regression analysis using morphometric measurement. A second model was derived from the regression analysis using skeletal measurements.

### First Model: Morphometric Measurements

Body size and shape (morphometric measurements) were used in regression analysis to predict body composition. The variables used in the analysis included body length, front height, rear height, thoracic circumference, pelvic circumference, front leg length, rear leg length, central foot pad length, central foot pad width, calcaneus width, head width, head circumference, facial length, and cranial length. Other variables included in the regression analysis were age, gender, and neuter status.

Stepwise multiple regression analysis was used to determine which morphometric variables provided the best estimate of lean body mass, fat mass, and percent body fat by DEXA. The data was analyzed with and without body weight as an independent variable. Models were developed for the entire study population and for two sub-populations, i.e., dogs with body weight less than or equal to 40 pounds and dogs with body weight greater than 40 pounds.

With all dogs included in the regression analysis and weight included as an independent variable, the best model that was derived to predict lean body mass included the following parameters: body weight (BW), cranial length (CL), cranial length*head circumference (CL=×HC), head width (HW), hind leg center foot pad length (HLCFPL), calcaneus width (CW), hind leg length (HLL), pelvic circumference (PC), and front height (FH). In this equation, with the lean body mass being represented by LBM:

LBM=(134.4×BW)−(1012×CL)+(23.5×(CL×HC))−(403.7×HW)+(319.74×HLCFPL)−(214.8×CW)+(1012.4×HLL)−(30.34×PC)−(119.4×FH)+2772.3. (1)

Applying this model to the entire study population predicted lean body mass correctly in 83% of the dog population (within ±10% of the DEXA value).

With all dogs included in the regression analysis and weight excluded as an independent variable, the best model that was derived to predict LBM included age, HLCFPL, PC, HC, front leg center foot pad width (FLCFPW), HLL, CL, and CL*HC. In this equation:

LBM=(122.5×age)+(174.33×HLCFPL)+(807.01×HLL)+(87.59×PC)−(570.1×HC)+(246.67×FLCFPW)−(2447×CL)+(58.92×(CL×HC))+10712. (2)

Applying this model to the entire study population predicted lean body mass correctly in 81% of the dog population (within ±10% of the DEXA value).

For more accurate equations under the first model, the dogs were split into groups of those with body weight less than 40 lbs. and those with body weight greater than 40 lbs. With all dogs having body weight less than 40 lbs. included in the regression analysis and weight included as an independent variable, the best model that was derived to predict LBM included age, BW, CL*HC, hind leg center food pad width (HLCFPW), CW, HLL and front leg length (FLL). In this equation:

LBM=(63.74×age)+(71.69×BW)+(5.31×(CL×HC))+(189.72×HLCFPW)−(122.8×CW)+(1019.6×HLL)−(337.7×FLL)−4148. (3)

Applying this model to the appropriate study population predicted lean body mass correctly in 100% of the respective dog population (within ±10% of the DEXA value).

With all dogs having body weight less than 40 lbs. included in the regression analysis and weight excluded as an independent variable, the best model that was derived to predict LBM included age, body length (BL), CL*HC, HLL, FLL and facial length (FL). In this equation:

LBM=(60.22×age)+(111.3×BL)+(7.61×(CL×HC))+(1401.6×HLL)−(480.2×FLL)−(169×FL)−5480. (4)

Applying this model to the appropriate study population predicted lean body mass correctly in 100% of the respective dog population (within ±10% of the DEXA value).

Similar techniques were applied to dogs with body weights greater than 40 lbs. With all dogs having body weight greater than 40 lbs. included in the regression analysis and weight included as an independent variable, the best model that was derived to predict LBM included age, BW, CL*HC, CL, HLCFPL, HLL, and FLL. This equation is given by:

LBM=(−146.1×age)+(104.71×BW)+(15.31×(CL'HC))−(675×CL)+(394.04×HLCFPL)+(1239.4×HLL)−(372.4×FLL)−6099. (5)

Applying this model to the appropriate study population predicted lean body mass correctly in 100% of the respective dog population (within ±10% of the DEXA value).

With all dogs having body weight greater than 40 lbs. included in the regression analysis and weight excluded as an independent variable, the best model that was derived to predict LBM included thoracic circumference (TC), PC, HLL, HLCFPL, FLL, and CL*HC. The equation is given by:

LBM=(148.92×TC)+(159.8×PC)+(944.01×HLL)+(679.12×HLCFPL)−(469.8×FLL)+(10.05×(CL×HC))−31075. (6)

Applying this model to the appropriate study population predicted lean body mass correctly in 95% of the respective dog population (within ±10% of the DEXA value).

Fat mass may be calculated in a similar manner. With all dogs included in the regression analysis and weight included as an independent variable, the best model that was derived to predict fat mass (FM) included BW, CL*HC, HLCFPL, HLL, and TC. This equation is given by:

FM=(272.41×BW)−(7.54×(CL×HC))−(208.8×HLCFPL)−(463×HLL)+(98.25×TC)+3110.3. (7)

Applying the model to the entire study population predicted FM correctly in 78% of the dog population (within ±10% of the DEXA value).

With all dogs included in the regression analysis and weight excluded as an independent variable, the best model that was derived to predict FM included TC, FLCFPL, and CW. This equation is given by:

FM=(366.14×TC)+(705.54×CW)−(365.1×FLCFPL)−18496. (8)

Applying this model to the entire study population predicted FM correctly in only 50% of the dog population (within ±10% of the DEXA value).

Dividing the dogs into two separate groups based on body weight for the prediction of fat mass was also beneficial, similarly to predicting lean body mass. With all dogs having body weight less than 40 lbs. included in the regression analysis and with weight included as an independent variable, the best model derived to predict FM included BL, HLCFPL, FLCFPW, PC, TC, and front height (FH). This equation is given by:

FM=(185.29×BL)−(193.5×HLCFPL)−(49.75×FLCFPW)+(79.99×PC)+162.51×TC−(49.72×FH)−9129. (9)

Applying this model to the appropriate study population predicted FM correctly in 100% of the respective dog population (within ±10% of the DEXA value).

With all dogs having body weight less than 40 lbs. included in the regression analysis and weight excluded as an independent variable, equation (9) was found to be the best model and the predicted values were found to be the same.

With all dogs having body weights greater than 40 lbs. included in the regression analysis and weight included as an independent variable, the best model that was derived to predict FM included BW, HLL, HLCFPL, FLL, and CL*HC. This equation is given by:

FM=(303.25×BW)−(917.6×HLL)−(339.5×HLCFPL)+(298.28×FLL)−(6.68×(CL×HC))+10067. (10)

Applying this model to the appropriate study population predicted FM correctly in 100% of the respective dog population (within ±10% of the DEXA value).

Similarly, with all dogs having body weights greater than 40 lbs. included in the regression analysis and weight excluded as an independent variable, the best model that was derived to predict FM included TC, PC, HLL, and CW. This equation is given by:

FM=(343.17×TC)+(234.01×PC)−(566.6×HLL)+(465.59×CW)−32291. (11)

Applying this model to the appropriate study population predicted FM correctly in 64% of the respective dog population (within ±10% of the DEXA value).

Percentage of fat may be calculated in a similar manner. With all dogs included in the regression analysis and weight included as an independent variable, the best model that was derived to predict percent fat (% Fat) included BL, RH, TC, HLL, CW, FLCFPW and HC. This equation is given by:

% Fat=(0.44×BL)+(0.34×RH)+(0.81×TC)−(4.2×HLL)+(0.95×CW)−(0.97×FLCFPL)−(1×HC)+47.87. (12)

Applying this model to the entire study population predicted % Fat correctly in 89% of the dog population (within ±10% of the DEXA value).

With all dogs included in the regression analysis and weight excluded as an independent variable, equation (12) was found to be the best model and the predicted values were found to be the same.

Dividing the dogs into two separate groups based on body weight for the prediction of percentage fat was similarly beneficial. With all dogs having body weight less than 40 lbs. included in the regression analysis and with weight included as an independent variable, the best model derived to predict % Fat included age, PC, and HW. This equation is given by:

% Fat=(1×PC)−(0.89×age)−(3.96×HW)+35.81. (13)

Applying this model to the appropriate study population predicted % Fat correctly in 79% of the respective dog population (within ±10% of the DEXA value).

With all dogs having body weight less than 40 lbs. included in the regression analysis and with weight excluded as an independent variable, equation (13) was found to be the best model and the predicted values were found to be the same.

With all dogs having body weight greater than 40 lbs. included in the regression analysis and with weight included as an independent variable, the best model derived to predict % Fat included BW, FLL, CL*HC, HLCFPL, and HLL. This equation is given by:

% Fat=(0.24×BW)+(0.96×FLL)−(0.01×(CL×HC))−(1.27×HLCFPL)−(2.62×HLL)+79.55. (14)

Applying this model to the appropriate study population predicted % Fat correctly in 100% of the respective dog population (within ±10% of the DEXA value).

With all dogs having body weight greater than 40 lbs. included in the regression analysis and with weight excluded as an independent variable, the best model derived to predict % Fat included PC and HLCFPL. This equation is given by:

% Fat=(0.34×PC)−(1.12×HLCFPL)+48.93. (15)

Applying this model to the appropriate study population predicted % Fat correctly in 86% of the respective dog population (within ±10% of the DEXA value).

### Second Model—Skeletal Measurement

Radiographic data provided skeletal size information that was used in regression analysis to predict lean body mass. From the head, ventral-dorsal, and lateral radiographic views, the following were measured: facial length, cranial length, skull width, pelvic length, pelvic width, tibia length, tibia diameter, calcaneus length, and length from calcaneal tuber to distal end of metatarsal bones. In addition to these variables, the following variables were also included in the regression analysis: cranial length×head width, pelvic length×pelvic width, tibia length×tibia diameter, tibia area, tibia circumference, tibia volume, tibia surface area, and tibia total area.

With all dogs included in the regression analysis and weight included as an independent variable, the best model that was derived to predict lean body mass included the parameters cranial length (cranL), calcaneus length (calL), and body weight. This equation is given by:

LBM=(165.42×BW)+(2993.72×calL)−(442.01×cranL)−4817.52. (16)

Applying this model to the entire study population predicted lean body mass correctly in 72% of the dog population (within ±10% of the DEXA value).

With all dogs included in the regression analysis and weight excluded as an independent variable, the best model that was derived to predict lean body mass included calL, head width (HW), and tibia area (TA). This equation is given by:

LBM=(3147.14×cal)+(1228.17×HW)+(24.39×TA)−17171.7. (17)

Applying this model to the entire study population predicted lean body mass correctly in only 47% of the dog population (within ±10% of the DEXA value).

Dividing the dogs into two separate groups based on body weight for the prediction of lean body mass was similarly beneficial. With all dogs having body weight less than 40 lbs. included in the regression analysis and with weight included as an independent variable, the best model derived to predict lean body mass included cranL, HW, BW, cranL×HW, pelvic length×pelvic width (PL×PW), and tibia circumference (TC). This equation is given by:

LBM=(−3842.51×cranL)−(2737.71×HW)+(85.48×BW)+(422.51×(cranL×HW))+(16.33×(PL×PW))+(77.37×TC)+23948.13. (18)

Applying this model to the appropriate study population predicted lean body mass correctly in 100% of the respective dog population (within ±10% of the DEXA value).

With all dogs having body weight less than 40 lbs. included in the regression analysis and with weight excluded as an independent variable, the best model derived to predict lean body mass included cranL×HW and calL. This equation is given by:

LBM=(50.38×(cranL×HW))+(2874.99×calL)−7205.82. (19)

Applying this model to the appropriate study population predicted lean body mass correctly in 57% of the respective dog population (within ±10% of the DEXA value).

With all dogs having body weight greater than 40 lbs. included in the regression analysis and with weight included as an independent variable, the best model derived to predict lean body mass included cranL, calL, and BW. This equation is given by:

LBM=(−734.02×cranL)+(3460.67×cal)+(169.43×BW)−4591.56. (20)

Applying this model to the appropriate study population predicted lean body mass correctly in 86% of the respective dog population (within ±10% of the DEXA value).

With all dogs having body weight greater than 40 lbs. included in the regression analysis and with weight excluded as an independent variable, the best model derived to predict lean body mass included HW and calL. This equation is given by:

LBM=(1513.35×HW)+(4790.33×calL)−23102.8. (21)

Applying this model to the appropriate study population predicted lean body mass correctly in 73% of the respective dog population (within ±10% of the DEXA value).

Notably, in the above-described manner, the best equation for the prediction of lean body mass using skeletal size data and body weight resulted in an r² of 0.99 and a predictability (±10%) of 100% for dogs less than or equal to 40 lbs. using 8 of the variables. These 8 variables were cranial length, head width, body weight, cranial length*head width, pelvic length*pelvic width, and tibia circumference. The best equation for the prediction of lean body mass using skeletal size data and body weight resulted in an r² of 0.99 and a predictability (±10%) of 86% for dogs greater than 40 lbs. using 3 variables, namely cranial length, calcaneus length, and body weight.

Similarly, the best equation for prediction of lean body mass using body size data, body weight, and age resulted in an r² of 0.99 and a predictability (±10%) of 100% for dogs less than or equal to 40 lbs. using 8 of the variables. These 8 variables included hind leg length, calcaneus width, hind leg central foot pad width, front leg length, cranial length*head circumference, body weight, and age. The best equation for prediction of lean body mass using body size data, body weight, and age resulted in an r² of 0.99 and a predictability (±10%) of 100% for dogs greater than 40 lbs. using 7 of the variables, namely hind leg length, hind leg central foot pad length, front leg length, cranial length, cranial length*head circumference, body weight, and age.

Likewise, the best equation for prediction of fat mass resulted in an r² of 0.99 and a predictability (±10%) of 100% for dogs less than or equal to 40 lbs. using body length, front height, thoracic circumference, pelvic circumference, hind leg central foot pad length, and front leg central foot pad width. The best equation for prediction of fat mass resulted in an r² of 0.97 and a predictability (±10%) of 100% for dogs greater than 40 lbs. using hind leg length, hind leg central foot pad length, front leg length, cranial length*head circumference, and body weight.

The results of this study proved remarkable. First, it was determined that correlation existed between physically measurable attributes and the percent of body fat in already obese dogs. This allowed the study to conclude that multiple regression analysis may be applied to specific categories of animals in order to determine which clinically measurable attributes most strongly correlate to an accurate prediction of fat mass or lean body mass. In effect, this type of analysis gives the animal practitioner a practical yet effective tool for devising an accurate food regimen and healthy diet for the animal.

### Example 2

Thirty-seven adult cats with body composition ranging from overweight to morbidly obese were evaluated. The following measurements and procedures were conducted: body weight, palpation and visual assessment, digital photographs (front, rear, side and from above), body size and shape measurements, radiographs (head, thoracic and pelvic) and DEXA.

Lean body mass, fat mass and percent body fat were determined by DEXA. This data was used to evaluate other methods by providing the dependent variables to predict body composition (lean body mass, fat mass and percent body fat) by using independent variables obtained from morphometric measurements, skeletal measurements, body weight, age, gender, and neuter status. In this manner, equations to predict lean body mass, fat mass, and percent of fat were derived. Two separate models were applied. The first model was derived from the regression analysis using morphometric measurement. A second model was derived from the regression analysis using skeletal measurements.

### First Model: Morphometric Measurements

Body size and shape (morphometric measurements) were used in regression analysis to predict body composition. The variables used in the analysis included body length, front height, rear height, thoracic circumference, pelvic circumference, front leg length, rear leg length, central foot pad length, central foot pad width, calcaneus width, head width, head circumference, facial length, and cranial length. Other variables included in the regression analysis were age, gender, and neuter status.

Stepwise multiple regression analysis was used to determine which morphometric variables provided the best estimate of lean body mass, fat mass, and percent body fat by DEXA.

With all cats included in the regression analysis, the best model that was derived to predict lean body mass included the following parameters: head circumference (HC), front leg length (FLL), front leg circumference (FLC), and hind leg central food pad width (HLCFPW). In this equation, with the lean body mass being represented by LBM:

LBM=(−5270)+(147×HC)+(248×FLL)+(317×FLC)−(103×HLCFPW). (22)

Fat mass may be calculated in a similar manner. With all cats included in the regression analysis and weight included as an independent variable, the best model that was derived to predict fat mass (FM) included body weight (BW), head circumference (HC), hind leg length (FILL), and front leg circumference (FLC). This equation is given by:

FM=(4910)+(438×BW)−(149×HC)−(296×HLL)−(206×FLC). (23)

### Second Model—Skeletal Measurement

Radiographic data provided skeletal size information that was used in regression analysis to predict lean body mass. From the head, ventral-dorsal, and lateral radiographic views, the following were measured: skull length, skull width, head length, head width, length from ileac crest to caudal edge of ischium, width from right to left ischitatic tuberosity, tibia length, tibia diameter, calcaneus length, and length from calcaneal tuber to distal end of metatarsal bones.

With all cats included in the regression analysis and gender included as an independent variable, the best model that was derived to predict lean body mass included the parameters: gender (G), head width (HW), pelvic length (PL), calcaneus length (calL), and calcaneal tuber length (calTL). This equation is given by:

LBM=−4630+301×G+358×HW+355×PL−2240×calL+871×calTL. (24)

1. A method of managing a weight condition in a companion animal comprising: using methods to determine the actual body percentage of body fat or lean body mass of a companion animal, using measurements of physical data of the companion animal to apply regression analysis based on the actual percentage of body fat or lean body mass, deriving one or more equations based on the regression analysis, to predict the percentage of body fat or lean body mass in the companion animal and using the predicted percentage of body fat or lean body mass to provide an effective weight loss regimen for the companion animal.

2. The method of claim 1 wherein the method to determine the actual body percentage or lean body mass of the companion animal is dual-energy X-ray absorptiometry.

3. The method of claim 1 wherein the companion animal is a cat.

4. The method of claim 1 wherein the one or more equations are two separate equations, wherein the first equation is used for a companion animal having a body weight equal to or less than a threshold amount, and the second equation is used for a companion animal having a body weight greater than a threshold amount.

5. The method of claim 4 wherein the companion animal is a dog and the threshold amount is forty pounds.

6. The method of claim 1, wherein the measurements of physical data comprise measurements of body weight (BW), cranial length (CL), cranial length*head circumference (CL×HC), head width (HW), hind leg center foot pad length (HLCFPL), calcaneus width (CW), hind leg length (HLL), pelvic circumference (PC), and front height (FH),
wherein the animal is a dog, and
wherein the equation used to predict lean body mass (LBM) is:
LBM=(134.4×BW)×(1012×CL)+(23.5×(CL×HC))−(403.7×HW)+(319.74×HLCFPL)−(214.8×CW)+(1012.4×HLL)−(30.34×PC)−(119.4×FH)+2772.3. (1)

7. The method of claim 1, wherein the measurements of physical data comprise measurements of age, hind leg center foot pad length (HLCFPL), pelvic circumference (PC), head circumference (HC), front leg center foot pad width (FLCFPW), hind leg length (HLL), cranial length (CL), and cranial length*head circumference (CL×HC),
wherein the animal is a dog and,
wherein the equation used to predict lean body mass (LBM) is:
LBM=(122.5×age)+(174.33×HLCFPL)+(807.01×HLL)+(87.59×PC)−(570.1×HC)+(246.67×FLCFPW)−(2447×CL)+(58.92×(CL×HC))+10712. (2)

8. The method of claim 5, wherein the animal is a dog with a body weight of less than 40 lbs and,
wherein the measurements of physical data comprise measurements of age, BW, CL*HC, hind leg center food pad width (HLCFPW), CW, HLL and front leg length (FLL),
and wherein the equation used to predict lean body mass (LBM) is:
LBM=(63.74×age)+(71.69×BW)+(5.31×(CL×HC))+(189.72×HLCFPW)−(122.8×CW)+(1019.6×HLL)−(337.7×FLL)−4148, (3)
or,
wherein the measurements of physical data comprise measurements of age, body length (BL), CL*HC, HLL, FLL and facial length (FL) and wherein the equation used to predict lean body mass (LBM) is:
LBM=(60.22×age)+(111.3×BL)+(7.61×(CL×HC))+(1401.6×HLL)−(480.2×FLL)−(169×FL)−5480 (4)
or,
wherein the measurements of physical data comprise measurements of cranial length (cranL), head width (HW), BW, cranial length×HW (cranL×HW), pelvic length×pelvic width (PL×PW), and tibia circumference (TC), and wherein the equation used to predict lean body mass (LBM) is:
LBM=(−3842.51×cranL)−(2737.71×HW)+(85.48×BW)+(422.51×(cranL×HW))+(16.33×(PL×PW))+(77.37×TC)+23948.13 (18)

9. The method of claim 5, wherein the animal is a dog with a body weight of more than 40 lbs and,
wherein the measurements of physical data comprise measurements of age, BW, CL*HC, CL, HLCFPL, HLL, and FLL and wherein the equation used to predict lean body mass (LBM) is:
LBM=(−146.1×age)+(104.71×BW)+(15.31×(CL×HC))−(675×CL)+(394.04×HLCFPL)+(1239.4×HLL)−(372.4×FLL)−6099 (5)
or,
wherein the measurements of physical data comprise measurements of age, thoracic circumference (TC), PC, HLL, HLCFPL, FLL, and CL*HC and wherein the equation used to predict lean body mass (LBM) is:
LBM=(148.92×TC)+(159.8×PC)+(944.01×HLL)+(679.12×HLCFPL)−(469.8×FLL)+(10.05×(CL×HC))−31075 (6)
or,
wherein the measurements of physical data comprise measurements of cranL, calcaneus length (calL), and BW, and wherein the equation used to predict lean body mass (LBM) is:
LBM=(−734.02×cranL)+(3460.67×calL )+(169.43×BW)−4591.56 (20)

10. The method of claim 1, wherein animal is a dog, and the measurements of physical data comprise measurements of BL, RH, TC, HLL, CW, FLCFPW and HC, and the equation used to determine body fat percentage (% fat) is:
% Fat=(0.44×BL)+(0.34×RH)+(0.81×TC)−(4.2×HLL)+(0.95×CW)−(0.97×FLCFPL)−(1×HC)+47.87. (12)

11. The method of claim 5, wherein the animal is a dog with a body weight of less than 40 lbs and,
wherein the measurements of physical data comprise measurements of age, PC, and HW, and
the equation used to determine body fat percentage (% fat) is:
% Fat=(1×PC)−(0.89×age)−(3.96×HW)+35.81 (13)

12. The method of claim 5, wherein the animal is a dog with a body weight of more than 40 lbs and,
wherein the measurements of physical data comprise measurements of BW, FLL, CL*HC, HLCFPL, and HLL, and the equation used to determine body fat percentage (% fat) is:
% Fat=(0.24×BW)+(0.96×FLL)−(0.01×(CL×HC))−(1.27×HLCFPL)−(2.62×HLL)+79.55, (14)
or
wherein the measurements of physical data comprise measurements of PC and HLCFPL, and the equation used to determine body fat percentage (% fat) is:
% Fat=(0.34×PC)−(1.12×HLCFPL)+48.93 (15)

13. The method of claim 1, wherein the animal is a cat, and,
wherein the measurements of physical data comprise measurements of head circumference (HC), front leg length (FLL), front leg circumference (FLC), and hind leg central food pad width (HLCFPW), and
wherein the equation used to predict lean body mass (LBM) is:
LBM=(−5270)+(147×HC)+(248×FLL)+(317×FLC)−(103×HLCFPW). (22)
or
wherein the measurements of physical data comprise measurements of gender (G), head width (HW), pelvic length (PL), calcaneus length (calL), and calcaneal tuber length (calTL),
and wherein the equation used to predict lean body mass (LBM) is:
LBM=−4630+301×G+358×HW+355×PL−2240×calL+871×calTL (24)

14. A method of managing a weight condition for a companion animal comprising determining the estimated body fat percentage of the companion animal and providing an effective weight loss regimen for the companion animal based on the estimated body fat percentage, wherein the body fat percentage determination comprises a visual or palpate assessment,
wherein the method further comprises an assessment of physical criteria observed during the visual or palpate assessment, with each assessment being assigned a particular number of points, and
wherein the number of points are combined to estimate the body fat percentage.

15. The method of claim 14 wherein the method further comprises determining an ideal body weight for the companion animal and providing a daily feeding regimen for the companion animal based on the ideal body weight.

16. The method of claim 14, wherein the method further comprises providing a food composition, wherein the food composition comprises protein, fat, fiber and carbohydrate.

17. The method of claim 14, wherein determining the estimated body fat percentage comprises biological information and measured physical criteria.

18. The method of claim 14, wherein determining the estimated body fat percentage of the companion animal comprises assessment of physical measurements of the companion animal.

19. The method of claim 14, wherein determining the estimated body fat percentage of the companion animal is accomplished through use of a spreadsheet, computer program, database or similar tool to receive input and to calculate the estimated percentage of body fat.

20. The method of claim 14, wherein the visual and palpate assessment includes determining the amount of face cover on the head and neck, prominence of bony structures in the face, distinction between the head and shoulder, scruff tightness and fat amount on neck, amount of pectoral fat, prominence and ease of palpation for the sternum, scapula and ribs, inguinal fat pad on the abdomen, ease of palpation of abdominal contents and overall body assessment including the shape from the side, shape from above, and stance.

21. A kit comprising in separate containers in a single package a (1) means for communicating information about or instructions for a method of assessing a companion animal comprising determining the estimated body fat percentage of the companion animal and providing an effective weight loss regimen for the companion animal as determined in any preceding claim, and (2) a food fat used to promote weight loss in the companion animal.
